(19) United States
(12) Patent Application Publication
(10) Pub. No.: US 2026/0241100 A1
(43) Pub. Date: Aug. 20, 2026

(54) ADAPTIVITY OF AUTOMATED MEDICAMENT DEVICE PARAMETER SETTINGS

(71) Applicant: INSULET CORPORATION, Acton, MA (US)

(72) Inventors: Joon Bok LEE, Acton, MA (US); William WHITELEY, Knoxville, TN (US); Yibin ZHENG, Hartland, WI (US); Jason O’CONNOR, Acton, MA (US); Anthony MEYER, Charlotte, NC (US)

(21) Appl. No.: 19/535,387

(22) Filed: Feb. 10, 2026

Related U.S. Application Data
(60) Provisional application No. 63/758,741, filed on Feb. 14, 2025.

(51) Int. Cl. A61M 5/172; A61M 5/142

(52) U.S. Cl. CPC A61M 5/1723; A61M 5/14248; A61M 2205/52; A61M 2230/201

(57) ABSTRACT

The disclosed examples describe different embodiments for customizing adaptivity of an automated medicament delivery (AMD) algorithm so as to optimize the algorithm more quickly and efficiently for an individual user as opposed to modifying the AMD globally in an attempt to optimize for all types of users.

### CROSS-REFERENCE TO RELATED APPLICATIONS

This application claims the benefit of and priority to U.S. Provisional Patent Application No. 63/758,741 filed on Feb. 14, 2025, which is incorporated herein by reference in its entirety.

### BACKGROUND

Adaptation of automated medicament delivery (AMD) systems to the insulin sensitivity of individual users is a critical part of ensuring effective glucose control outcomes. However, applying a constant rate of adaptation across all users may result in anti-adaptive behaviors for some users, including possibility of adaptation of system behaviors leading to potentially worsened outcomes compared to prior iterations (including initial conditions). Typically, AMD systems are initialized to a population average value or a user input value, and then may be further adapted to the individual user based on available glucose and/or insulin delivery trajectories over time. However, this may be suboptimal because it does not consider the potential impact of new insulin delivery history that is sub-optimal to the user's glucose control outcomes, which may lead to a worsening of the AMD system performance over time if such outcomes are persistent.

It would be beneficial if a confidence level of the insulin delivery history was calculated for each new insulin delivery based on glucose control outcomes, instead of using a constant factor.

In other instances, AMD systems may be utilized by people who have advanced Type-2 diabetes and are insulin dependent. However, insulin is just one of many medications that these users incorporate into their diabetes care, and additional medicaments may also be utilized-including certain medicaments, such as GLP-1, that may dramatically impact the user's bodyweight and thus change the insulin sensitivity of such users far beyond the extent that would be affected by minor changes in a user's lifestyle and/or growth. An AMD system that is tuned to respond conservatively to changes in insulin needs will lead to significant risk of potential hypoglycemia until it is fully adapted.

It would be beneficial if an AMD system was operable to adjust for dramatic changes in life patterns of Type-2 diabetic users based on an increased number of occurrences of medicament suspension, such as insulin suspension.

AMD systems typically incorporate an initial estimate of the user's average insulin requirements when first initiating insulin therapy for its users, then execute an adaptation of its delivery estimates over time. However, certain adaptivity schemes in AMD systems may lead to a sub-optimal convergence behavior, where the system attempts to further adapt its behavior to edge case outcomes even after strong outcomes (e.g., exceptional time-in-range percentages, or the like) are achieved. Such implementations may lead to an oscillatory adaptation behavior, where a more aggressive adaptation may worsen an initially positive control outcome (e.g., substantial or near exceptional time-in-range percentages) over time.

It would be beneficial if a system augmented the adaptivity of AMD systems to incorporate an assessment of prior glucose control outcomes, and provide the capability to revert to those settings if those prior outcomes are significantly better than the current outcomes.

AMD algorithms typically adapt their insulin delivery recommendations based on an estimate of the total daily insulin requirements for each user. However, these adaptations typically lead to continuously increasing insulin needs for users with high insulin resistance, as increasing insulin deliveries by AMD systems do not result in significant changes in glucose control outcomes for these users. In these cases, an increase in insulin delivery may result in increased vulnerability of the users to the side effects of insulin (such as weight gain) without significant benefits in glucose control outcomes.

In these instances, it would be beneficial if a system evaluated a user's steady state glucose outcomes versus insulin delivery requirements, and recommended reduction of the estimated insulin needs for the user over time if there is minimal change in glucose outcomes despite wide variations in the amounts of insulin delivered between time periods.

### BRIEF SUMMARY

Disclosed is a system that may include a memory and a processor coupled to the memory. The memory is configured to store user history for a user, where the user history includes medicament delivery data and sensor data of the user over a plurality of days. The processor may be operable to analyze the medicament delivery data and the sensor data for each day in a portion of the user history, assign a weight value to each day from the portion of the user history that includes medicament delivery data and/or sensor data meeting a weighting criteria, determine a new parameter for estimating medicament delivery dosages using additional data from each day assigned a weight and the assigned weight value assigned to the respective day, generate an estimated medicament delivery dosage for delivery to the user using the new parameter, and cause delivery of the estimated medicament delivery dosage.

Disclosed is a method that may include determining a number of medicament suspension that occurred over a predetermined period. A new medicament delivery parameter may be weighted based on the number of medicament suspension. An updated medicament delivery parameter may be determined using the weighted new medicament delivery parameter and a prior medicament delivery parameter, and delivery of medicament may be estimated based on the updated medicament delivery parameter.

Disclosed is system that may include a memory and a processor. The memory may be configured to store user history for a user, where the user history includes an operational status of a medicament delivery device over a period of time. The processor may be operable to determine a number of suspensions of medicament delivery from the operational status of the medicament delivery device. The processor, based on the determined number of suspensions, may adjust a learning rate of an adaptivity algorithm for adapting a medicament delivery parameter, and determine the medicament delivery parameter using the adjusted learning rate for the adaptivity algorithm.

Disclosed is a system that may include a memory and a processor. The memory may be configured to store user history for a user, where the user history includes an indication of suspension of delivery of medicament over a period of time. The processor may be operable to generate a trust index for a new user history for a period of time. The trust index may be a value indicating how much the new user history is to be compared to a previous user history. The processor may determine an enhanced medicament delivery parameter that incorporates the trust index as an additional factor to a number of days and a fixed weighting value, use the enhanced medicament delivery parameter to determine a basal delivery dosage of the medicament. The processor may cause the basal delivery dosage to be delivered by a medicament delivery device.

Disclosed is a system that may include a memory and a processor. The memory may be configured to store user history for a user, where the user history includes glucose concentrations over a period of time. The processor may be operable to receive an indication that a new total daily dosage is an increase over a prior total daily dosage, evaluate a change in mean glucose concentrations between adaptivity cycles, and, based on a result of the evaluation determining being an increase of a second mean glucose concentration from a first mean glucose concentration, update the prior total daily dosage using a portion of the new total daily dosage to provide an updated total daily dosage.

### BRIEF DESCRIPTION OF THE DRAWINGS

To easily identify the discussion of any particular element or act, the most significant digit or digits in a reference number refer to the figure number in which that element is first introduced.

FIG. 1 illustrates an exemplary process for establishing an adaptivity level of confidence according to the disclosed subject matter.

FIG. 2A illustrates an exemplary process for adjusting to dramatic changes in life patterns of a user according to the disclosed subject matter.

FIG. 2B illustrates an exemplary process for adapting a medicament delivery parameter according to the disclosed subject matter.

FIG. 3 illustrates an exemplary process to reduce risk of reduction in glucose outcomes due to sub-optimal adaptation rates in automated medicament delivery systems according to the disclosed subject matter.

FIG. 4 provides graphics illustrating a problem to be solved by the disclosed subject matter.

FIG. 5 illustrates an example process to reduce medicament delivery when adapting to a user's medicament needs in an AMD systems as shown in FIG. 4 according to the disclosed subject matter.

FIG. 6 illustrates an exemplary medicament system operable to implement the example processes disclosed herein.

### DETAILED DESCRIPTION

In typical embodiments, AID systems may be initialized to a population average value or a user input value, and may be further adapted to the individual user based on available glucose and/or insulin delivery trajectories over time. In an example, the confidence in a current TDI represented by TDIₖ) may be captured as follows:

$$T D {I}_{k} - ( 1 - R \cdot {N}_{k} ) \cdot {TDI}_{k - 1} + R \cdot {N}_{k} \frac{{\sum }_{j = 1}^{{T}_{end}} I ( j )}{\frac{{T}_{end}}{288}} , \qquad Equation 1$$

where Nₖ is the number of days of new data available at the time of k^{th} adaptivity execution, I(j) is the insulin delivery history available for the k^{th} adaptivity execution, R is the rate of adaptivity, and the term T_{end} is a tunable parameter. Adaptivity rate R may, for example, refer to the extent to which the TDI estimate is updated based on the average daily insulin requirements obtained from a new insulin delivery history. In an example, the value of the adaptivity rate R may be a fixed value, such as 0.2 or the like, which corresponds to weighing the previous TDI estimate (represented by TDIₖ₋₁) by 40%, and weighing the current average daily insulin needs by 60%, if, for example, 3 days of data are available (i.e., Nₖ=3). The maximum value of R may be considered to be related to the maximum number of days of new data, where at most the sum of new days of data will not drive the coefficient of the first term in this equation to below 0. For instance, Nₖ may be at most 10, in which case, R should not be greater than 0.1 such that the weighting of the previous TDI estimate does not become 0. A rule may be expressed as:

$$0 \leq R \leq \frac{1}{{N}_{k}}$$

However, such implementation incorporates the user's new insulin delivery history without consideration of the glucose control outcomes based on the new insulin delivery history. For instance, the user may have received a significantly reduced total daily insulin (TDI) in the most recent iteration, leading to increased rate of hyperglycemia. The significantly reduced TDI may then be a value that is incorporated into the adaptation by the AID system, which can lead to erroneous or less than optimal results.

Here, the TDI, or total daily insulin value, may be adapted over time as controlled by the adaptivity rate R (ranging from 0 to 1), where the adaptivity rate R represents the extent to which the TDI estimate is updated based on the average daily insulin requirements from new insulin delivery history.

The foregoing may be implemented using equations that may take a number of different forms and be implemented in a number of different ways.

For example, in typical AMD systems, the algorithm may adapt to the user's insulin needs by reviewing the user's previous insulin delivery history. In one example, such adaptation may be implemented as TDI_{updtd}, which is a weighted average of the user's previous and current total daily insulin needs:

$${TDI}_{updtd} ( i ) = X \cdot TDI ( i - 1 ) + ( 1 - X ) \cdot TDInew ( i ) \qquad Equation 2$$

where TDI_{updtd} is a weighted sum of a latest TDI for a current operational cycle TDI_{new} TDI(i−1) is a TDI for a prior cycle. The variable X is the rate at which an AMD algorithm may adapt.

Here, the user's previous TDI estimate (i.e., TDI(i−1)) and a current, newly available TDI (i.e., TDI_{new}) may be added as a weighted sum governed by the term X. A typical value for X may be 0.8 per day (or 20% based on the latest TDInew) and 80% based on the old TDI (i.e., TDI(i−1)). If X were 0, TDI_{updtd} would fully ignore old TDI values (TDI(i−1) and use the current, newly available TDI (i.e., TDI_{new}) to be fully adapted.

However, as described in the following example, these terms TDI_{updtd}, TDI_{new}, and TDI(i−1) may be tuned to be applicable to a wide range of use cases, and, accordingly, may not adapt sufficiently rapidly to address risks of hypoglycemia in cases where the user may experience very fast changes in their insulin needs, such as when a user with T2 diabetes begins medications, such as GLP-1.

Also disclosed is a method to assess the user's steady state glucose outcomes versus insulin delivery requirements, and provide recommendations for reduction of the estimated insulin needs for the user over time if there is minimal change in glucose outcomes despite wide variations in the amounts of insulin delivered between time periods. For example, in typical embodiments, an AMD system adapts its estimate of a user's insulin needs based on past insulin delivery history using equations, such as the following:

$${TDI}_{new}^{′} = ( 1 - 0.2 \cdot {N}_{days} ) {TDI}_{days} + 0.2 \cdot {N}_{days} {TDI}_{new} \qquad Equation 3$$

where the new TDI estimate, TDI′_{new}, is a weighted sum of the previous TDI estimate (TDI_{old}) and the total daily insulin within the newly available insulin delivery history (TDI_{new}—for TDI current), weighted by the duration of the new history in number of days (N_{days}), where the number of days (N_{days}) does not exceed 5.

In the following examples, the rate of adaptivity, in some examples, may be viewed as a degree to how much the new data is trusted as compared to the old data. The value of the weighting applied to the new data indicates a level of trust in the new data versus the old data. For example, the higher the value of the weighting applied to the data, the greater the level of trust. Different criteria may be used to determine how much new data is to be weighted versus the weighting of old data. In some systems, the new data is simply trusted 100% in lieu of the old data. However, 100% trust in new data may be misplaced, and may result in frequent adaptations. Hence, the examples herein are directed to not adapting frequently, but to learning more rapidly based on the user history data, so the adaptivity rate may also be considered a learning rate. When new data is weighted more and trusted more, the rate of adaptivity and the learning rate are higher.

The following discussion establishes a “level of confidence” in the outcome of a current adaptivity rate in an AMD system, and provides the system an option to revert, or even “Reset” its adaptivity to prior settings if the level of confidence is sufficiently low over its course of adaptation.

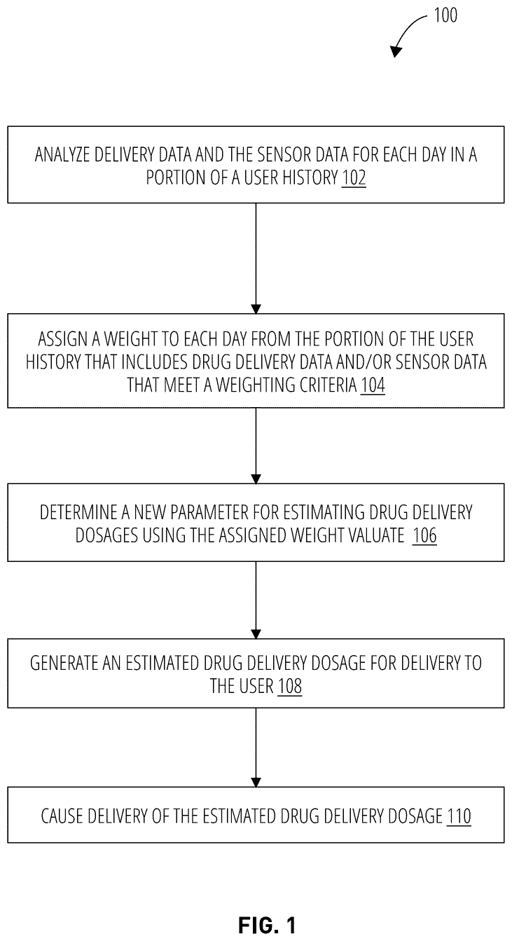

FIG. 1 illustrates an exemplary process for establishing an adaptivity level of confidence according to the disclosed subject matter.

The process 100 may be implemented by a processor and an ongoing confidence level of new insulin delivery may be calculated for each new insulin delivery history based on glucose control outcomes. Specifically, as explained in more detail below, the weighted sum of past insulin delivery history for TDI may be captured based on glucose control outcomes, and the avoidance of utilizing outdated sums may be established by executing a hard cut-off on the number of days of history that are being utilized. This is fully ignoring the previous adaptivity shown in Equation 1 and moving toward continuous adaptation based on weighting as described in more detail below.

In block 102, the processor executing the process 100 may be operable to analyze the medicament delivery data and the sensor data for each day in a portion of the user history. “User history” may refer to medicament delivery data and/or sensor data of a user per day (or on a daily basis) over a number of days as well as daily data that is derived from the medicament delivery data and/or sensor data for respective days of the number of days. For example, the user history may include estimates of the total daily dosage (TDD), which for insulin is referred to as TDI, over the past several months, weeks or days. For example, the past N number of TDI estimates over a fixed unit of time (e.g., 14 days, 10 days, or the like) or no older than a threshold (e.g., 4 days, 7 days, 14 days, 24 days, 1 month, or the like), may be evaluated based on their respective associated glucose control outcomes (i.e., time in range (TIR)). A “good” glucose control outcome would be a high percentage of time (e.g., greater than 70%, greater than or equal to 68%, 80%, or the like). While the “best” glucose control outcomes are the top 5, 7, 10, 15 or 20 glucose control outcomes that satisfy the “good” criteria.

In block 104, the process 100 assigns a weight to each day from the portion of the user history that includes medicament delivery data and/or sensor data that meet a weighting criteria. In some examples, the weighting criteria is a data value that exceeds or is less than a threshold value, and the threshold value depends upon a specific category of the medicament delivery data and/or, for sensor data, a specific data value or a range of data values. For example, the weighting may be a fixed weighting for each day—with the highest weighting applied to the day with the best time in range. This may be captured in an operational example with reference to Table 1 below.

In this example, a user history and assigned weights may look as follows:

TABLE 1

| | | | Weight (must sum |
|---|---|---|---|
| Date | TIR % 70-180 | TDI [U] | up to 1.0 or 100%) |
| August 13 | 69 | 74.7 | 0 |
| August 14 | 73 | 78.4 | 0.1 |
| August 15 | 76 | 81.7 | 0.4 |
| August 16 | 80 | 74.4 | 0.5 |

| | |
|---|---|
| New TDI for August 17 | 77.7 U |

In Table 1, the user's history data includes the user's time in range (TIR) and the user's TDI (in Units) and is used to calculate a new TDI for use in a future day, which in this example is the next day—August 17. In the example, there are 4 days of data entries—August 13 through August 16—from the user's history data. The date with the lowest percentage of time in range—August 13—is dropped by the processor and not used in the calculation of a new TDI. The processor may be operable to generate a weighting for each of the remaining days with the highest weighting (e.g., 0.5) being assigned to the date with the highest percentage of time in range (TIR) (e.g., 80%—August 16) and the next highest weighting (e.g., 0.4) being applied to the next highest percentage TIR (e.g., 76%—August 15), and the last weighting (e.g., 0.1) being applied to the last remaining day that has a percentage TIR (e.g., 73%—August 14). In this example, the new TDI for August 17 has a high “confidence level” because of the effects of the weights assigned over the three days with the best glucose control outcomes, resulting in a weighted TDI value of 77.7U that prioritizes days with higher time in range percentages.

In block 106, the process 100 may determine a new parameter for estimating medicament delivery dosages. The processor may use assigned weights and apply the weighting to the TDI for the respective day. For example, the weight 0.5 may be applied (e.g., as a multiplier) to the TDI 74.4U to arrive at a weighted TDI value of 37.2 for August 16, the weight 0.4 may be applied (e.g., as a multiplier) to the TDI 81.7U to arrive at a weighted TDI value of 32.7 for August 15, the weight 0.1 may be applied (e.g., as a multiplier) to the TDI 78.4U to arrive at a weighted TDI value of 7.8 for August 14. The processor may be operable to sum the weighted TDI values for each day to produce a new parameter, such as a new TDI (i.e., 37.2U+32.7U+7.8U=77.7U). In this example, the new parameter was the New TDI value 77.7U.

The above example was provided for ease of discussion, but the system also envisions a user history having a greater number of days. In an example of a greater number of days of user history, the past 14 days, or 2 weeks, of TDI estimates may be evaluated once per day, with a weighting applied for each day of the user history—with the highest weighting applied to the day with the highest percentage of time in range. For example, a two-week period of time may be used where there are 14 days of data entries. The algorithm may be operable to discard the 4 days of the 14 days that have the lowest percentage time in range. The remaining 10 days may be weighted with the day having the highest percentage time in range receiving the greatest weighting and the day with the lowest percentage time in range receiving the lowest weighting. The weightings may be, for examples values of 0.04, 0.05, 0.06, 0.07, 0.08, 0.09, 0.10, 0.11, 0.12, 0.13, and 0.15, where the sum of the weightings in 1.0 or 100%. The weighting 0.04 is the lowest, while the weighting 0.15 is highest.

In block 108, the process 100 generates an estimated medicament delivery dosage using the weighted new medicament delivery parameter and a prior medicament delivery parameter for delivery to the user. The process 100 causes delivery of the estimated medicament delivery dosage, at 110.

Below is a general equation that implements the above process regardless of the number of days of user history:

$${TDI}_{N} = {\sum }_{k = 1}^{X} {TDI}_{N - k} {W}_{N - k} \qquad Equation 4$$

where N is the current day or a future day, k is a day before the current day, W is the weighting value, and X is the number of days of user history.

While the above references were made to TDI as a medicament delivery parameter, other medicament delivery parameters may include at least one of the TDI, a total daily dosage, a cost function ratio, basal rate, a glucose set value, an insulin-to-carbohydrate ratio, or a duration of insulin action.

In an alternate embodiments, the weight of the individual TDI values may instead be calculated to scale based on the percentage % time in range (TIR) against a time in range threshold, such as at least 65%, with larger values above the threshold providing a greater weighting, such as the following:

$${W}_{N} = \frac{max ( {TIR}_{N} - {TIR}_{th} , 0 )}{{TIR}_{target} - {TIR}_{th}} \qquad Equation 5$$

where W_{N} is a weighting for a day N, TIR_{N} is the time in range for the day N, the TIRₜₕ is the time in range threshold, the TIR_{target} is the time in range target setting.

In an example, the TIR_{target} may be set as 70%, and TIRₜₕ may be set as 65%. Equation 2 may be applied to a number of days in the user history. The value of the weights may be based on the number of days selected to be evaluated. However, the weights may be further scaled in such a way that the sum of the weights for all the considered TDI data add up to 100%. The higher weighting W_{N} applies to the day with the higher TIR.

In further alternate embodiments, the weighting may be set such that only the TDI values for which a TIR above a specific threshold were achieved may be considered, such as a threshold of 70%, 75%, 55%, or the like.

By using Equation 3, a controller may be operable to perform continuous weighting.

It is envisioned that a change in TDI by a few units may impact glucose control outcomes, but that is just from one day to the next, and will make a significant difference overall.

In yet a further embodiment, it is envisioned that the adaptation (i.e., TDIₖ) from Equation 1 may be combined with the adaptation (i.e., TDI_{N}) from the Equation 2-using a weighted average, average, or the like, for example.

Alternatively, the above Equations 4 and 5 could be other applied parameters other than TDI, such as, for example, a basal parameter, the cost parameters Q and R, target set points, and the like. For instance, the cost parameters may be similarly adjusted, as in the following equations A and B:

$${Q}_{N} = {\sum }_{k = 1}^{X} {Q}_{N - k} {W}_{N - k} \qquad Equation A$$

Where the weight may instead be modified by the prior values of the cost parameters, such as following:

$${W}_{N} = \frac{max ( {Q}_{N} - {Q}_{th} , 0 )}{{Q}_{tarqet} - {Q}_{th}} \qquad Equation B$$

Additional parameters may be adjusted for automated delivery algorithms for any other medicaments, such as the generalized total proportion of delivery that would be delivered for constant infusion requirements (basal insulin delivery in case of insulin deliveries), the ratio of infusion amounts to compensate for specific disturbances (Insulin-to-carbohydrate ratios, and the like for insulin deliveries), or others.

This example provides processes for AMD systems to review whether there are increases in the number of suspensions of medicament delivery versus the earlier uses of the user's systems, and, in response to there being an increased number of suspensions of medicament delivery, especially insulin, increase the AMD algorithm's rate of adaptivity.

The typical adaptivity algorithm may be tuned to be applicable to a wide range of use cases, and thus may not adapt sufficiently rapidly to address risks of hypoglycemia in cases where the user may experience extremely fast changes in their insulin needs, such as when a user with T2 diabetes begins medications, such as GLP-1. For example, the use of GLP-1 may dramatically impact the user's bodyweight and thus change the insulin sensitivity of such users far beyond the extent that would be affected by minor changes in a user's lifestyle and/or physical growth. To address this risk, in an embodiment, an AMD system may execute the following process to adjust the AMD algorithm by increasing the AMD algorithm's rate of adaptivity, particularly when there is an apparent decrease in their insulin needs.

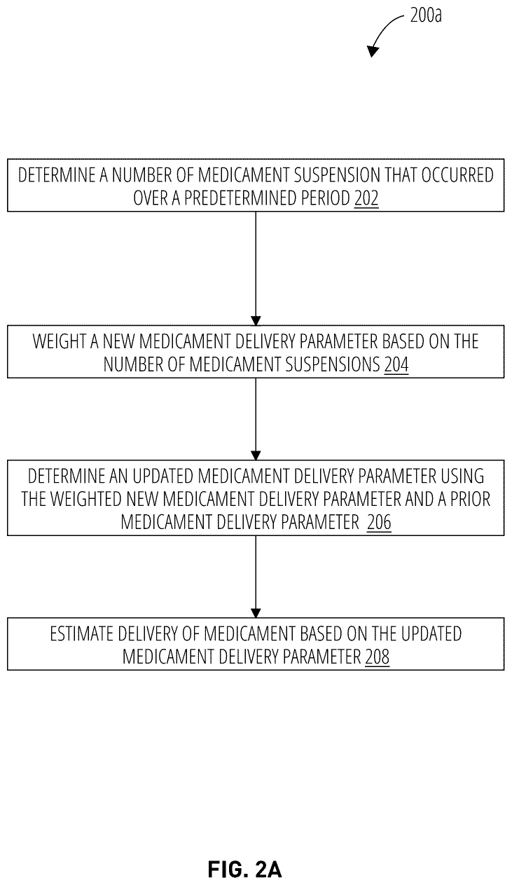

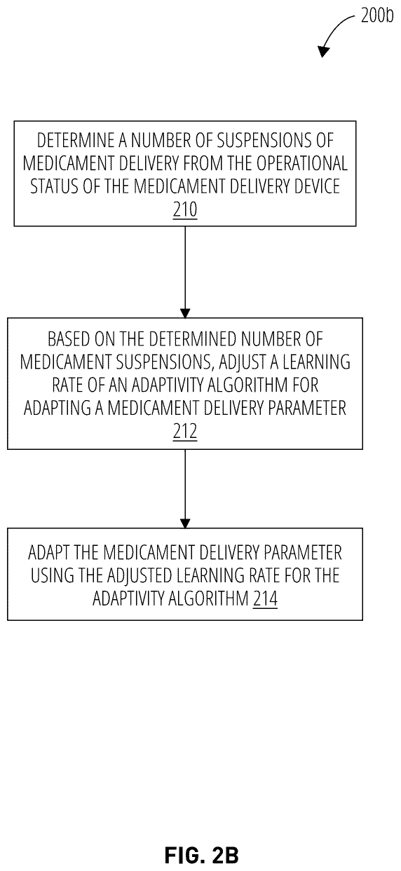

FIG. 2A illustrates an exemplary process for adjusting to dramatic changes in life patterns of a user according to the disclosed subject matter. Although the example process 200a depicts a particular sequence of operations, the sequence may be altered without departing from the scope of the present disclosure. For example, some of the operations depicted may be performed in parallel or in a different sequence that does not materially affect the function of the process 200a. In other examples, different components of an example device or system that implement the process 200a may perform functions at substantially the same time or in a specific sequence. While the example is directed to insulin suspensions, the adaptivity rate adjustment process 200a is applicable to other types of medicament, such as GLP-1 or the like.

The process 200a of FIG. 2A may be implemented as part of an AMD algorithm that is configured to determine different medicament delivery parameters, track user physiological conditions, such as glucose levels, ketone levels, heart rate, glucose control outcomes, or the like, and store in a memory user history related to medicament delivery, the tracked user physiological conditions, and the like.

At a high level, the process 200a is directed to an adjustment may be executed by reviewing periods of insulin delivery, outside of impact of user delivered boluses, where there has been a dramatic change in the system behavior. Specifically, it is expected that a user with a dramatic reduction in insulin needs may experience much greater periods of insulin suspension. Therefore, if the periods of insulin suspension are above a threshold, the TDI adaptation performed by the AMD algorithm may be accelerated or modified to fully restart the adaptivity cycle with the current TDI estimate. The process 200a is an example implementation of the concept of adaptivity rate adjustment.

The adaptivity rate adjustment may be based on an operational cycle of the medicament delivery device. In an example, the operational cycle is an amount of time that an AMD system (e.g., a sensor, a medicament delivery device, and an AMD algorithm) takes to receive sensor data and deliver a predetermined amount of a medicament. In another example, the operational cycle is a series of operations performed by the component parts of an AMD system. For example, the series of operations may be obtaining and/or receiving sensor data (e.g., a glucose measurement value) from a sensor, generating a medicament delivery dosage, and sending a signal to a medicament delivery device to expel the medicament delivery dosage.

The user history, in addition to the operational status, may include medicament delivery data and sensor data of the user over the period of time. For example, the processor may analyze medicament delivery data and sensor data of the user for each day in a portion of a user history. In this example, the user history may also include an operational status of the medicament delivery device. The operational status may include indications of a normal operational status, a suspended operational status (i.e., medicament suspension), nighttime operational, meal operational status, or the like.

In block 202 of the process 200a, the processor determines a number of medicament suspensions that have occurred over a predetermined period. For example, a processor may analyze a user history to determine a number of medicament suspensions that occurred over a predetermined period. In an example, “medicament suspension” refers to a complete stoppage of delivery of medicament for an operational cycle of the system, and an example of an operational cycle is when the AMD algorithm receives a glucose level from a continuous glucose monitor, and delivers medicament to the user. A manual bolus is not considered to be a medicament suspension. The predetermined period of time may be a lifecycle of a wearable medicament delivery device, 12 hours, 24 hours, 48 hours, or the like.

The determination of whether the user has an increasing number of insulin delivery suspensions may be obtained by the processor from a user delivery history. The user delivery history is a collection of data that includes information related to delivery of medicament to the user (e.g., number of units of medicament delivered daily, medicament suspensions, bolus dosages (calculated and delivered, correction boluses, rescue boluses)), glucose measurement values, user modifications to basal rate, and the like. The user delivery history may be stored in a memory accessible to a processor executing the AMD algorithm. The process 200a is operable to use the information in the user delivery history to not only adapt, but to increase the rate of adaptivity so an optimal result can be provided to the user more quickly.

In this example, a medicament suspension, in this case, an insulin suspension, may be determined according to the following:

$${I}_{susp} ( {i}_{j} - k ) = \{ \begin{matrix}1 & I ( {i}_{j} - k ) = 0 \\ 0 & otherwise\end{matrix} \qquad Equation 6$$

where the variable Iₛᵤₛₚ represents whether insulin has been suspended for this operational cycle, and I represents the amount of insulin delivered for the operational cycle iⱼ-k. When I(iⱼ-k) is equal to zero (meaning no insulin is delivered), Iₛᵤₛₚ is equal to 1, which is interpreted as a medicament suspension for that operation cycle.

The processor may be operable to sum the number of insulin suspensions within a predetermined time period L_{new} as follows:

$${N}_{susp} ( i ) = {\sum }_{k = 1}^{{L}_{new}} {I}_{susp} ( {i}_{j} - k ) \qquad Equation 7$$

where Nₛᵤₛₚ is the #of cycles of suspensions (could be equated to the duration of suspensions) and Iₛᵤₛₚ is a counter; when there is a suspension, this value is 1 so Nₛᵤₛₚ increments, for example, by 1. The predetermined period of time L_{new} may be a time period at which a wearable medicament delivery device is replaced, such as 72 hours or the like.

Once the number of medicament suspensions within the predetermined time period is determined. Specifically, it is expected that a user with dramatic reduction in insulin needs may experience a much greater number of occurrences of insulin suspension. Therefore, the processor may determine if the number of insulin suspension is above a threshold value (e.g., 8, 12, 24, or the like). If the determination is Yes, the number of insulin suspension is above a threshold value, the TDI adaptation may be accelerated or modified to fully restart the adaptivity with the current TDI estimate. With the indication that the number of medicament suspensions exceeds the threshold value, the process 200a may proceed to block 204.

At block 204, a processor executing the process 200a weights a new medicament delivery parameter based on the number of medicament suspensions. There are a number of methods for determining the weighting but the following equation, Equation 8, determines the weighting based on whether the number of medicament suspensions is increasing (and the user's insulin needs are decreasing).

The following is but one equation that may be used to generate a new medicament delivery parameter, TDI_{updtd}(i):

$${TDI}_{updtd} ( i ) = max ( \frac{1}{2 - min ( max ( \frac{{N}_{susp} ( i )}{{N}_{susp} ( i - 1 )} , 1 ) , 1.5 )} X , 0 ) \cdot {TDI}_{new} ( i ) + min ( 1 , 1 - \frac{1}{2 - min ( max ( \frac{{N}_{susp} ( i )}{{N}_{susp} ( i - 1 )} , 1 ) , 1.5 )} X ) \cdot TDI ( i - 1 ) \qquad Equation 8$$

where X is a constant weighting factor (e.g., 0.2, 0.25, or the like), Nsusp(i−1) is a number of medicament suspensions from the previous operational cycle, and Nsusp(i) is the number of medicament suspensions for the current operational cycle.

In Equation 8, if the latest Nₛᵤₛₚ (i.e., Nₛᵤₛₚ(i)) is increasing), Equation 8 weights the TDI_{new}(i) (i.e., the new TDI) more heavily than the TDI(i−1) (i.e., the previous TDI) to arrive at TDI_{updtd}(i). Specifically, the adaptivity, as shown in Equation 8, is bounded such that any reduction in suspension will not result in more rapid adaptivity, but an increase in suspension results in an increase in the rate of adaptivity, up to two times the original rate. An additional limit is applied to the overall adaptivity, based on the value of X, such that the TDI estimate is at most capped at 100% trust in the new TDI history (i.e., TDI_{new}(i)). Note that TDI is a medicament delivery parameter where the higher the value, the more aggressive the AMD algorithm behaves in trying to enforce compliance with other settings, such as target glucose set point.

In block 206, the processor executing the process 200a may be operable to determine an updated medicament delivery parameter (e.g., TDI_{new}(i)) using the weighted new medicament delivery parameter and a prior medicament delivery parameter. In an example, the updated medicament delivery parameter is at least one of total daily insulin, a total daily dosage, a cost function ratio, basal rate, a glucose set value, insulin-to-carbohydrate ratio, or duration of insulin action.

In block 208, the process 200a estimates delivery of medicament based on the updated medicament delivery parameter. For example, an updated medicament delivery parameter, such as TDI_{new}(i), may be used to determine a medicament delivery dosage, which may be a basal dosage, a bolus dosage, or a combination of both.

In some examples, this disclosed adjustment may be executed by reviewing periods of insulin delivery, outside of the impact of user delivered boluses, where there has been a dramatic change in the system behavior. Even when the dramatic change is a result of the user now exercising on a regular basis and forgetting to enter Activity Mode or the like, for example, the disclosed embodiment is operable to accommodate that situation as well as situations where the user starts a new medication, such as GLP-1, or the like.

In this example as well as others, adaptation is described with reference to TDI adaptation. However, the described adaptation may be related to any total daily dosage (TDD) of medicament. With reference to diabetes treatment, medicament delivery parameters other than TDI may be adapted, such as basal rate, setpoint, duration of insulin action (DIA), Q:R ratio, insulin-to-carbohydrate (IC) ratio, and the like.

It should be noted that the algorithm, in some examples, adapts in favor of increased number of suspensions, and the adaptivity is bounded such that any reduction in suspension does not result in more rapid adaptivity, but an increase in suspension does result in an increase in the rate of adaptivity, for example, up to two times the original adaptivity rate. The foregoing adaptation is directed to a reduction in TDI and a focus on limiting hypoglycemia risk. Accordingly, it does not specifically address situations where a user stops exercising and the AMD algorithm needs to be adapted back to delivering more insulin (due to a reduction in suspensions).

FIG. 2B illustrates an exemplary process for adapting a medicament delivery parameter according to the disclosed subject matter. Although the exemplary process 200b depicts a particular sequence of operations, the sequence may be altered without departing from the scope of the present disclosure. In examples, new data may be rated higher when it comes to adaptivity rate for an AMD algorithm. In this example, the rate of adaptivity may be linearly scaled based on the ratio of the number of suspended cycles in the newest available history, versus the number of suspended cycles in the previously available history.

A system that may implement the process 200b may include a processor and a memory. The memory may be configured to store a user history for a user. The user history may include, for example, an operational status of a medicament delivery device over a period of time. The processor is coupled to the memory, and is operable to access the user history stored in the memory. According to some examples, the process 200b causes a processor to be operable to determine a number of suspensions of medicament delivery from the operational status of the medicament delivery device at step 210. The user history may, for example, in addition to the operational status, includes medicament delivery data of the medicament delivery device and sensor data of the user over the period of time.

Continuing with the example, the processor, when determining a number of suspensions of medicament delivery from the operational status of the medicament delivery device, is further operable to evaluate insulin delivery by a medicament delivery device to determine the number of suspensions of insulin delivery. For example, the medicament delivery device may be operable to access the memory and evaluate the stored user history for the number of suspensions of medicament delivery.

At step 212, the process 200b may include adjusting a learning rate of an adaptivity algorithm for adapting a medicament delivery parameter based on the determined number of medicament suspensions. The adapted medicament delivery parameter may be one or more of: a total daily insulin, a cost function ratio, a basal rate, a glucose set value, insulin-to-carbohydrate ratio, a duration of insulin action, or the like for use in a current iteration of the adaptivity algorithm.

According to some examples, the process 200b may further include adapting the medicament delivery parameter using the adjusted learning rate for the adaptivity algorithm at step 214. In addition, the processor when adapting the medicament delivery parameter, may be further operable to use the medicament delivery data and the sensor data to determine a new medicament delivery parameter, and retrieve an old medicament delivery parameter.

Additionally, the processor may be further operable to input the new medicament delivery parameter and the old medicament delivery parameter into a current iteration of the adaptivity algorithm. The processor, in an example, is further operable to linearly scale the learning rate based on a ratio of the number of suspensions of medicament delivery in a newest available user history versus a number of suspensions of medicament delivery in a previous history available prior to the newest available user history.

Other technical features may be readily apparent to one skilled in the art from the following figures, descriptions, and claims.

In typical embodiments, AID systems may automatically adjust their insulin delivery settings over time based on a new user history that includes newly available glucose and/or insulin delivery history. As mentioned with respect to FIG. 1, this adjustment may be based on the average total daily insulin delivery, such as shown in Equation 1.

Similar to the earlier examples, there are a number of ways that the adaptivity of Equation 1 is used. For example, a trust index for the user's time in range may be established via Equation 1.

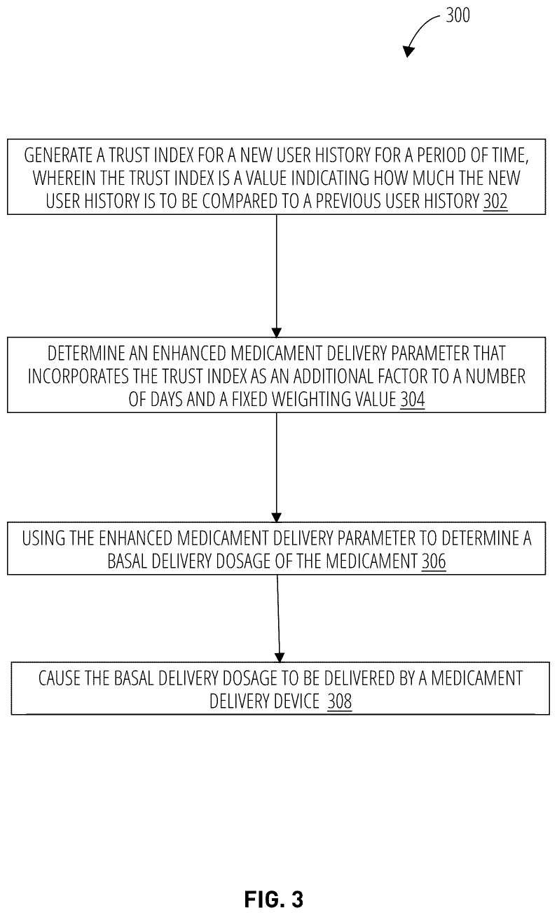

FIG. 3 illustrates an exemplary process to reduce risk of reduction in glucose outcomes due to sub-optimal adaptation rates in automated medicament delivery systems according to the disclosed subject matter.

A trust index may be created as a metric based on how much the user's time in range changed since the last calculation of the trust index. The trust index metric is compared to an expected rate of improvement, such as 2%, or 0.02). If the rate of improvement was less than the expected rate of improvement, the new user history may be weighted as low a zero (0), which means there is no change to the total daily insulin quantity. If the rate of improvement has accelerated more rapidly than the threshold, the trust index may result in up to a full trusting of the new insulin delivery history with a zero weight applied to the previous input total daily insulin quantity. Alternatively, a minimum rate of adaptivity, such as 0.1, may still be applied despite the rate of regression. A different relative ratio or threshold may also be applied. Of course, the trust index metric may be generated based off of other parameters instead of time in range (TIR). For example, the trust index may be based on mean glucose measurement values, glucose control outcomes, and the like. A glucose control outcome may refer to a glucose measurement value, values or parameters based on a glucose measurement value, such as time in range, or the like that are due to AMD algorithm changing a dosage of medicament to be delivered.

In some examples, the trust index is used to adjust the adaptivity rate based on an indication, or a determination, of how much the new data is trusted. The higher the trust index value the more the algorithm trusts the new history as compared to the old history, and therefore, the new history is weighted more in the calculation of the total daily insulin.

However, such an implementation of completely trusting medicament delivery parameters generated only using a new user history may incorporate the user's new insulin delivery history without consideration of the glucose control outcomes based on the new insulin delivery history. For instance, the user may have received a significantly reduced total daily insulin in the most recent iteration, leading to increased rate of hyperglycemia, which may then be a value that is incorporated into the adaptation by the AID system.

In one proposed embodiment, the AID system's adaptation may be modulated by the change in glucose control outcomes in the new cycle. Specifically, the rate of adjustment may be modified or reduced if there has been a reduction in glucose outcomes associated with the new insulin delivery history, which may be captured as described with reference to Equation 1 above, while the trust index Tₖ is calculated as shown in Equation 5 below:

$${T}_{k} = min ( max ( 0 , \frac{{TIR}_{k - 1} - {TIR}_{k - 2}}{2} ) , \frac{1}{R \cdot {N}_{k}} ) \qquad Equation 9$$

where Nₖ is the number of days of new data available at the time of k^{th} adaptivity execution, R is the rate of adaptivity (i.e., adaptivity rate), TIRₖ−1 and TIRₖ−2 are time in range for the previous two adaptation cycles.

In an example, the rate of adaptivity may be modified by a trust index, Tₖ, which compares a metric, such as the time in range based on the current insulin delivery history with the time in range from the prior insulin delivery history or a prior day or the like. The comparison may be further compared against threshold, such as an expected rate of improvement, such as 2% or the like. In the proposed embodiment, if the rate of improvement regressed, the trust index may result in a weighting of the new history to as low as 0, resulting in no change to input total daily insulin quantity. If the rate of improvement has accelerated than the threshold, the trust index may result in up to a full trusting of the new insulin delivery history with a 0 weight applied to the previous input total daily insulin quantity. Alternatively, a minimum rate of adaptivity, such as 0.1, may still be applied despite the rate of regression. A different relative ratio or threshold may also be applied.

In alternate proposed embodiments, a record of prior total insulin delivery amounts, and glucose control outcomes, may also be assessed. For instance, an AID system may compile a record of prior daily insulin delivery and glucose control outcomes. At any adaptation cycle, the AID system may apply a weighted average of the “known best” total daily insulin delivery settings (i.e., the TDI settings that resulted in the highest percentage time in range for a time period, in other words, the best TDI settings) and the most recent insulin delivery history, to calculate the next insulin delivery setting, such as the following:

$${TDI}_{k} = ( 1 - R \cdot {N}_{k} \cdot {T}_{k}^{′} ) \cdot {TDI}_{best} + R \cdot {N}_{k} \cdot {T}_{k} \cdot \frac{{\sum }_{j = 1}^{{T}_{end}} I ( j )}{\frac{{T}_{end}}{288}} \qquad Equation 10$$

$${T}_{k}^{′} = min ( max ( 0 , \frac{{TIR}_{k - 1} - {TIR}_{best}}{2} ) , \frac{1}{R \cdot {N}_{k}} ) \qquad Equation 11$$

In one proposed embodiment, the history of best TDI and TIR values may be assessed over a moving window, with considerations only being applied to up to 3 months before the current time.

In Equations 10 and 11, where TIRₖ−1 is a most recent time-in-range value for a last adaptation cycle. Nₖ is the number of days of new data available at the time of k^{th} adaptivity execution, and R is the rate of adaptivity (i.e., adaptivity rate). TDI_{best} is a value that represents the TDI of the periods of time that had the best time in range (TIR_{best}). If the time in range from the latest (i.e., new) user history is better than the TIR_{best}, then the new history is trusted more than the TDI_{best}. In this example, the trust index value T′ₖ is determined using Equation 11.

The higher the trust index value T′ₖ, the greater the trust in the latest, or new user history.

The trust index T′ₖ may be used to adjust the adaptivity rate, or learning rate, instead of utilizing a constant value for the adaptivity rate (e.g., R), which is a fixed rate of learning. In some examples, the trust index T′ₖ replaces the adaptivity rate/learning rate R.

In block 302, process 300 generates a trust index for a new user history for a period of time, wherein the trust index is a value indicating how much the new user history is to be compared to a previous user history. In block 304, process 300 determines an enhanced medicament delivery parameter that incorporates the trust index as an additional factor to a number of days and a fixed weighting value. In block 306, process 300 using the enhanced medicament delivery parameter to determine a basal delivery dosage of the medicament. In block 308, process 300 causes the basal delivery dosage to be delivered by a medicament delivery device.

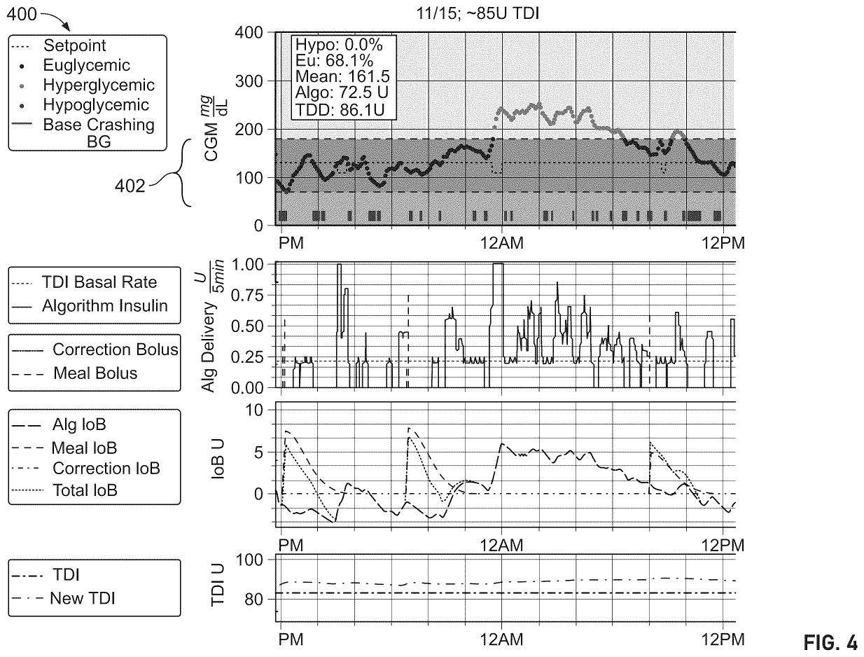

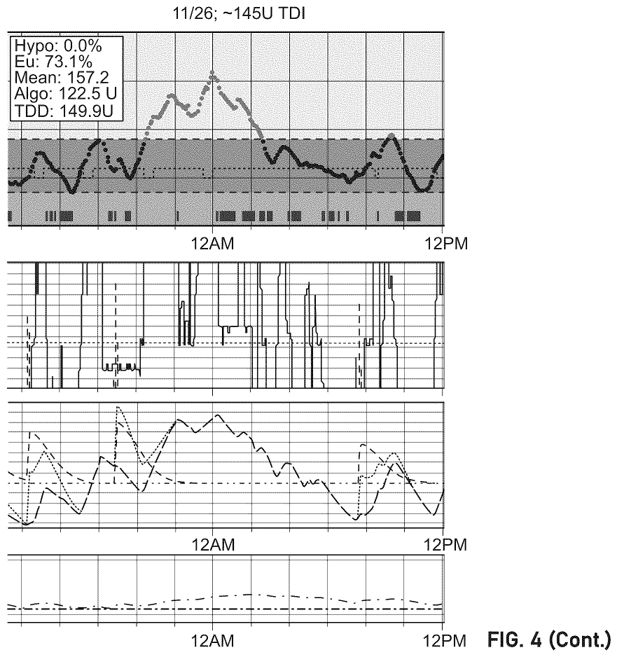

FIG. 4 provides graphics illustrating a problem to be solved by the disclosed subject matter.

The data presented in FIG. 4 illustrates that, for some users, the user's time in range (TIR) is not substantially affected by the user's total daily dosage (TDD), whether 85U or 145U, the TIR is substantially the same. As shown in the graphs of the two-day glucose data 400 of FIG. 4, the same user receives a TDD of approximately 85U on the left figure for one day (i.e., 11/15), versus a TDD of approximately 145U in the right figure for another day (i.e., 11/26). Despite these drastically different total insulin dosages between the respective days, the total TIR of the safe glycemic range (70-180 mg/dL) differs by only approximately 5% between the two days as shown in the two respective time in range 402 sub-boxes in the glucose concentration plots for each respective day shown in each respective graph 11/15 and 11/26. With such an outcome, it is wasteful to administer the excess units (i.e., over the 85U for day 11/15) of insulin, or any other medicament, when a lesser number of units of medicament achieves substantially the same TIR result. In addition, an increase in medicament delivery may result in increased vulnerability of the user to possible side effects of medicament (such as weight gain) without any significant benefits in glucose control outcomes (e.g., improved time in range (TIR)). In these examples, the medicament may include insulin, GLP-1, glucagon, and the like.

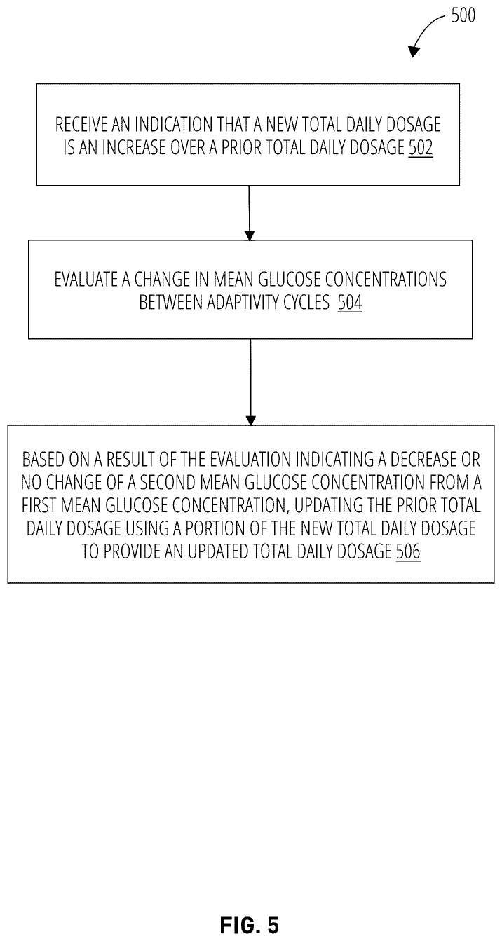

The following describes an exemplary system and an exemplary process, such as process 500 of FIG. 5, that evaluates this excess usage of medicament. A system, for example, may be configured to evaluate the usage of medicament based on how much the user's mean glucose measurement values are changing over a period of time with reference to a glucose threshold (e.g., 15 mg/dL, 20 mg/dL, 45 mg/dL, or the like). The period of time may, for example, be an adaptation cycle. An adaptation cycle may refer to a lifecycle of a medicament delivery device. For example, an adaptation cycle may start when a medicament delivery device is prepared, applied to a user, and starts managing a user's glucose, and may end when that medicament delivery device is no longer able to manage the user's glucose because, for example, the medicament reservoir is empty, a power supply fails, or the like. During the adaptation cycle, the AMD algorithm may update one or more medicament delivery parameters, such as TDD, total daily insulin (TDI), insulin-to-carbohydrate ratio, insulin sensitivity, insulin on board, or the like.

An AMD system may adapt its estimate of a user's insulin needs based on past insulin delivery history by calculating medicament delivery parameters using equations, such as the following:

$${TDI}_{new}^{′} = ( 1 - 0.2 \cdot {N}_{days} ) {TDI}_{old} + 0.2 \cdot {N}_{days} {TDI}_{new} \qquad Equation 12$$

where the new TDI estimate, TDI′_{new}, is a weighted sum of the previous TDI estimate (TDI_{old}) and the total daily insulin within the newly available insulin delivery history (TDI_{new}—for TDI current), weighted by the duration of the new history in number of days (N_{days}).

For example, as the user continues to receive more insulin, the system continues to adapt to a higher insulin estimate for the user's total daily insulin values. However, in certain populations that utilize AMD systems, a user may be highly insulin insensitive to changes in insulin delivery—that is, the user may not experience significant changes in their glucose outcomes when increasing insulin delivery. For instance, on the 11/15 graphic of FIG. 4, the time in range 402 (as shown by the line that remains between the highest dashed horizontal line and the lowest dashed horizontal line) for a type-2 diabetic user who has a TDI of approximately 85Units is shown and the 11/26 (right side) graphic of FIG. 4 shows a TDI of approximately 145Units for the same user, who happens to have substantially the same amount of time in range. In this example, this user experiences similar times in range despite receiving 60U greater daily insulin from one day to another (i.e., 85U versus 145U), as shown in FIG. 4, which may be an indication of insulin insensitivity.

In these instances, the drastic increase in insulin delivery may not be beneficial to the user, as the tradeoffs between slight changes in TIR may not, for example, outweigh the significant increase in the probability of the user experiencing side effects due to the increased medicament dose.

Instead, as shown in the following example of FIG. 5, a decaying factor that reduces medicament delivery if there is minimal change in TIR outcomes may be applied into any upward adjustments in TDI values.

FIG. 5 illustrates an example process 500 for implementing a process to reduce medicament delivery when adapting to a user's medicament needs in an AMD systems as shown in FIG. 4 according to the disclosed subject matter.

The processor executing process 500, at block 502, receives an indication that a new total daily dosage is an increase over a prior total daily dosage. For example, the processor, when evaluating a change in mean glucose concentrations between adaptivity cycles, is operable to compare the prior total daily dosage to the new total daily dosage. The processor, based on a result of the evaluation indicating the new total daily dosage being greater than the prior total daily dosage, generates the indication that the new total daily dosage is an increase over the prior total daily dosage. In this example, if the total daily dose that may be adapted by the AMD system would increase, the change in mean glucose concentrations may also be assessed.

In block 504, the processor evaluates a change in mean glucose concentrations between adaptivity cycles. In an example, the processor may determine whether the mean glucose concentration has decreased by more than a threshold, such as 20 mg/dL, between the previous, k−1th adaptation cycle, and k−2^{nd} adaptation cycle, or the like. In an example, if the amount of change does not meet the exemplary 20 mg/dL threshold, the processor may determine there has been no change.

In block 506, the processor, based on the result of the evaluation indicating a decrease or no change of a second mean glucose concentration from a first mean glucose concentration, updating the prior total daily dosage using a portion of the new total daily dosage to provide an updated total daily dosage. For example, if the mean glucose concentration has decreased by more than a threshold, such as 20 mg/dL, between the previous, k−1th adaptation cycle and k−2^{nd} adaptation cycle, then the full 100% of the new total daily dosage (e.g., TDI_{new} of Equation 14 below) in insulin delivery requirements may be applied. In this instance, the portion of the new total daily dosage may be 100%. However, if the mean glucose has not changed or decreased, only a much smaller proportion of the adjustment to the total daily dosage (e.g., TDI_{new} of Equation 14 below) may be used in the total daily dosage adjustment, such as 20% of the change in insulin delivery may be applied. While, in this instance, the portion of the new total daily dosage may be less than 100%.

Alternatively, at block 504, the processor may generate an indication that the new total daily dosage is a decrease from the prior total daily dosage based on the new total daily dosage being lesser than the new total daily dosage. Instead of proceeding to block 506, the process 500 would instead implement a typical adaptivity algorithm that utilizes the TDI_{new} in Equation 12 to calculate an updated total daily dosage. Note that Equation 12 represents a standard adaptivity rate calculation.

The above described process 500 may be implemented using a decaying factor that reduces insulin delivery if there is minimal change in TIR outcomes may be applied into any upward adjustments in TDI values.

For example, the newest TDI value, TDI_{new}′, is determined. However, the newest TDI may not be fully trusted, i.e., not weighted 100%, and may be reduced by a percentage. So, a TDI_{new} may be calculated to using an equation, such as Equation 13 below, that only trusts the newest TDI value, for example, only 20% (i.e., 0.2):

$${TDI}_{new} - ( 1 - 0.2 \cdot {N}_{days} ) {TDI}_{o l d} + 0.2 \cdot {N}_{days} {TDI}_{new}^{′} \qquad ( Equation 13 )$$

$${TDI}_{new}^{′} = ( 1 - 0.2 \cdot {N}_{days} ) {TDI}_{o l d} + 0.2 \cdot {N}_{days} {TDI}_{new}$$

where the new TDI estimate, TDI′_{new}, is a weighted sum of the previous TDI estimate (TDI_{old}) (which may be the most recent, previous TDI estimate) and the total daily insulin within the newly available insulin delivery history (TDI′_{new}) for the current TDI estimate, weighted by the duration of the new history in number of days (N_{days}), where the number of days (N_{days}) does not exceed 5.

Continuing with the example, it is determined whether TDI′_{new}>TDI_{old}. If TDI′_{new} is greater than TDI_{old}, this may be captured as follows in Equation 14:

$${TDI}_{n e w}^{″} = \{ \begin{matrix}\begin{matrix}{TDI}_{old} + ( {TDI}_{new}^{′} - {TDI}_{old} ) \cdot \\ max ( min ( 1 , \frac{\overline{{CGM}_{k - 2}} - \overline{{CGM}_{k - 1}}}{20} , 0.2 )\end{matrix} & {TDI}_{new}^{′} > {TDI}_{old} \\ {TDI}_{new}^{′} & otherwise\end{matrix}$$

(Equation 14) TDI″_{new} is a revised TDI′_{new} that enables the reduction of medicament delivery when mean glucose measurement values remain within a threshold. In Equation 14, CGMₖ₋₁ and CGMₖ₋₂ are the mean glucose measurement values from the previous two adaptation cycles (e.g., the previous two wearable medicament delivery devices), the factor TDI′_{new} is the TDI calculated using Equation 13, which uses the newly available, or latest, insulin delivery history TDI estimate (TDI_{new}), and TDI_{old} is the previous TDI estimate, or, specifically, the most recent, previous TDI estimate.

The application of Equation 14 has conditions as shown by the conditional inequality TDI′_{new}>TDI_{old}, if the TDI estimate TDI′_{new} (as calculated using Equation 13) is greater than (>) TDI_{old}, then Equation 14 is implemented and the TDI estimate to be used for adaptation is TDI′_{new}, calculated using the formula shown beside the inequality; otherwise (i.e., TDI_{old} is greater than TDI′_{new}), TDI′_{new} as calculated by Equation 13 is used as TDI′_{new}.

Using Equation 14, a processor is operable to address the deficiencies caused by a user's high insensitivity to insulin described above, if the updated TDI′_{new} or, more generally, total daily dose (TDD), that may be adapted by the AMD system may cause an increase in TDI, the change in mean glucose concentrations may also be assessed. In an example, the mean glucose concentration CGMₖ₋₂ may be a first mean glucose concentration, and the CGMₖ₋₁ may be a second mean glucose concentration. In an example, the first mean glucose concentration CGMₖ₋₂ is a mean of glucose measurement values from a previous adaptation cycle, and the second mean glucose measurement value CGMₖ₋₁ is a mean of glucose measurement values from the latest adaptation cycle that is later than the previous adaptation cycle.

In an example, if the mean glucose concentration has decreased (i.e., the difference between the mean values of CGMₖ₋₂ and CGMₖ₋₁ is negative) by more than a threshold, such as 20 mg/dL shown in the denominator of the mean glucose concentration factor of Equation 14 above, between the previous (k−1th) adaptation cycle and (k−2nd) adaptation cycle, then the full 100% of the total daily dosage may be applied. In the exemplary Equation 14, the threshold is shown as 20 mg/dL, but another threshold, such as 15 mg/dL or 30 mg/dL may be chosen. If the mean glucose concentration has decreased by less than a threshold, a lower proportion, such as 80%, proportional to amount that has decreased versus the threshold, may also be applied.

This proportion may be constrained to a threshold of maximum and minimum values, such as 20% and 100%.

Alternatively, rather than limiting of the maximum adjustment to 100% of the differences, a positive change in mean glucose concentration within the threshold may be instead scaled by the amount of increased insulin delivery that would be considered by the adaptivity scheme, through various methods, such as a linear scaling.

In Equation 13, however, if the mean glucose has not changed or decreased, only much smaller proportion of this adjustment, such as 20% (represented by the 0.2 in the maximum function of the above equation), of the total daily dosage may be applied.

A positive change in mean glucose concentration within the threshold may scale the amount of increased insulin delivery that would be considered by the adaptivity scheme, through various methods, such as a linear scaling.

In an alternate example, a reduction in TDI estimate may be continuously applied as long as the glucose control outcomes, such as TIR or mean glucose concentration, has not been significantly altered. In one example, this may be applied as an additional factor as follows:

$${TDI}_{new} = ( 1 - 0.2 \cdot {N}_{days} ) {TDI}_{old} + 0.2 \cdot {N}_{days} ( {TDD}_{new} \cdot min ( 1 , 0.99 + 0 \text{.01} \cdot \frac{\vert {TIR}_{N - 1} - T I {R}_{N - 2} \vert}{0.5} ) ) \qquad ( Equation 15 )$$

where TIR_{N-1} represents a time in range for a prior period of N, where N is the current day, in this case, TIR_{N-1} is one day prior to the current day N, and TIR_{N-2} represents the time in range for a day two days prior to the current day N.

In this example of Equation 15, the total daily dose may be based on newly adapted insulin delivery history, TDI_{new}, which may be adjusted to between 99% and 100% of the true value depending on the net change in TIR of the user's glucose control outcomes. A difference of less than 0.5% (represented by the 0.5 in the denominator) in TIR results in a scaling reduction in the full amount of TDI to be adjusted, with a difference in TIR of 0% resulting in a full 1% reduction in the final TDI amount to be incorporated. an adjustment of.

In this example, Equation 15 is biased to reduce medicament (e.g., insulin) over time. For example, the values 0.99+0.01 and the denominator 0.5 may be changed to decay more slowly or more rapidly as the case may require.

Other technical features may be readily apparent to one skilled in the art from the following figures, descriptions, and claims.

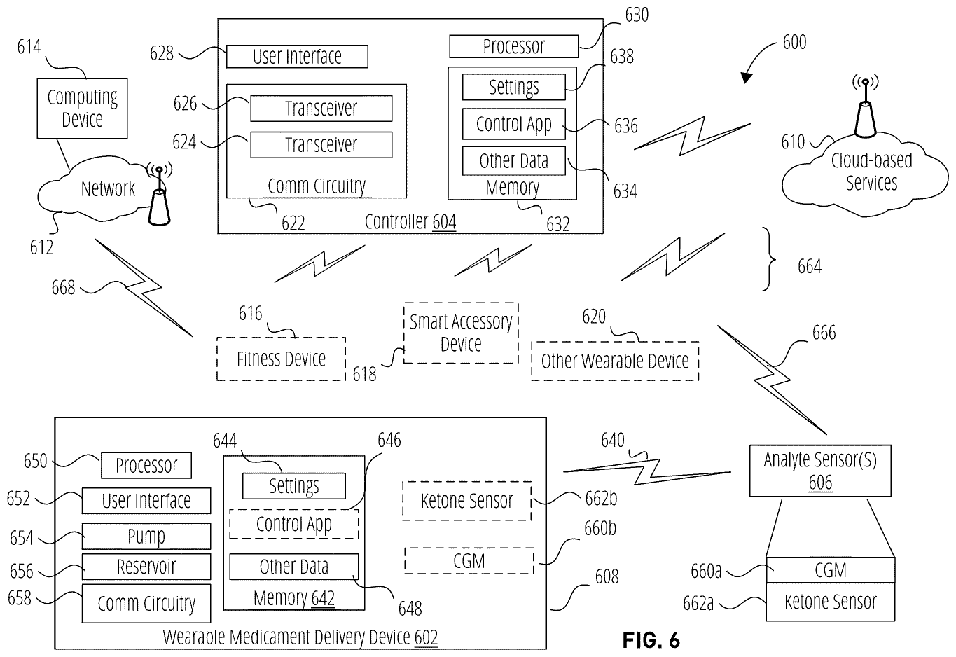

FIG. 6 illustrates an exemplary automated medicament delivery system operable to implement the example processes disclosed herein.

The automated medicament delivery system 600 is suitable for delivering a liquid medicament, such as insulin to a user in accordance with the disclosed embodiments. The medicament system 600 may include a wearable medicament delivery device 602, a controller 604 and an analyte sensor(s) 606.

The medicament delivery system 600 includes a wearable medicament delivery device 602, a controller 604, an analyte sensor(s) 606, a housing(s) 608, a cloud-based services 610, a network 612, a computing device 650, a fitness device 616, a smart accessory device 618, another wearable device 620,

The controller 604 may include processor 630, a memory 632, communication circuitry 622, and a user interface 628. The communication circuitry 622 may include transceiver 624 and transceiver 626. The memory 632 may store other data 634, a control application 636, and settings 638.

The wearable medicament delivery device 602 may include a memory 642, a processor 650, a user interface 652, a pump 654, a reservoir 656, and a communication circuitry 658. The memory 642 may store settings 644, a control application 646, and other data 648.

The analyte sensor(s) 606 may include a continuous glucose monitor 660a and a ketone sensor 662a.

The medicament delivery system 600 may communicate via a number of communication links such as: communication link 640, a communication links 664, a communication link 666, and a communication link 668.

The wearable medicament delivery device 602 may also optionally include a continuous glucose monitor 660b and/or a ketone sensor 662b.

The wearable medicament delivery device 602 may be a device that is worn on the body of the user. In an example, the wearable medicament device 602 may be directly coupled to a user (e.g., directly attached to skin of the user via an adhesive, or the like). In an example, a surface of the wearable medicament device 602 may include an adhesive to facilitate attachment to the skin of a user.

The wearable medicament device 602 may include a processor 650. The processor 650 may be implemented in hardware, software, or any combination thereof. The processor 650 may, for example, be a microprocessor, a logic circuit, a field programmable gate array (FPGA), an application specific integrated circuit (ASIC) or a microprocessor coupled to a memory, such as memory 642. The processor 650 may maintain a date and time as well as be operable to perform other functions (e.g., calculations or the like). The processor 650 may be operable to execute a control application 646 stored in the memory 642 that enables the processor 650 to direct operation of the wearable medicament device 602. The control application 646 may control insulin delivery to the user per an AMD control approach via an AMD algorithm as describe herein. For example, the control application 646 may be the AMD algorithm. The memory 642 may hold settings 644 for a user, such as AMD algorithm settings, such as maximum insulin delivery, insulin sensitivity settings, total daily insulin (TDI) settings, target glucose settings, and the like. The memory 642 may also store other data 648, such as ketone values, total daily insulin values, glucose measurement values from analyte sensor(s) 606 or controller 604, insulin dosage amounts (both basal and bolus) that have been delivered to a user (e.g., expelled by the pump 654) and the like from previous minutes, hours, days, weeks, or months or operation of one or more wearable medicament delivery devices 602.

The analyte sensor(s) 606 may be operable to collect physiological condition data, such as ketone values, ketone values with a time stamp, glucose measurement values (also referred to herein as “blood glucose values” or “blood glucose”), glucose measurement values and a timestamp, and the like. In some examples, the ketone values and/or the glucose values are shared with the wearable medicament device 602, the controller 604, or both. In an additional example, the analyte sensor(s) 606 may include multiple sensors, such as a continuous glucose monitor 660a and a ketone sensor(s) 662a. In a further example, the wearable medicament device 602 may optionally include a continuous glucose monitor 660b and a ketone sensor 662a, which may be removably incorporated or fully integrated within the wearable medicament device 602. For example, the continuous glucose monitor 660a and the ketone sensor 662b may be incorporated in one or more housings 608 of the wearable medicament device 602. Note that ketones may also be detected using a breath sensor (which is not shown but may be incorporated in the controller 604) or urine content sensor and stored in the respective memories 632 and 642 via a user input of the ketone levels; however, a subcutaneous ketone sensor(s) gives more accurate information and is more continuous. As described herein, the MDA and AMD system is described based on receiving ketone values received subcutaneously. Use of a ketone breath sensor or urine sensor as part of or in addition to the analyte sensor(s) 606 may delay receipt of the ketone values and modifications to the system 600 may be made.

For example, the communication circuitry 658 of the wearable medicament device 602 may be operable to communicate with the analyte sensor(s) 606 and the controller 604 as well as the devices 616, 618, and 620. The communication circuitry 658 may be operable to communicate via Bluetooth, cellular communication, near field communication (NFC) and/or other wireless protocols. While not shown, the memory 642 may include both primary memory and secondary memory. The memory 642 may include random access memory (RAM), read only memory (ROM), optical storage, magnetic storage, removable storage media, solid state storage or the like.

The wearable medicament device 602 may include a reservoir 656. The reservoir 656 may be operable to store liquid medicaments, medicaments, or therapeutic agents suitable for automated delivery, such as, insulin, glucagon-like peptide-1 (GLP-1) receptor agonist, pramlintide, glucose-dependent insulintropic polypeptide (GIP), other hormones, or co-formulations of two or more of glucagon, GLP-1, pramlintide, and insulin; as well as pain relief medicaments, such as opioids or narcotics (e.g., morphine, or the like), methadone, blood pressure medicines, chemotherapy medicaments, fertility medicaments, or the like.

A fluid path to the user may be provided via tubing and a needle/cannula (not shown). The fluid path may, for example, include tubing coupling the wearable medicament device 602 to the user (e.g., via tubing coupling a needle or cannula to the reservoir 656). The wearable medicament device 602 may be operable based on control signals from the processor 650 to expel the liquid medicaments, medicaments, or therapeutic agents, such as insulin, from the reservoir 656 to deliver doses of the medicaments, medicaments, or therapeutic agents, such as the insulin, to the user via the fluid path. The processor 650 may be operable to cause insulin to be expelled from the reservoir 656.

There may be one or more communications links 664 with one or more devices physically separated from the wearable medicament device 602 including, for example, a controller 604 of the user and/or a caregiver of the user and/or a sensor(s) 606. The communication links 664 may include any wired or wireless communication link operating according to any known communications protocol or standard, such as Bluetooth®, NFC, Wi-Fi, a near-field communication standard, a cellular standard, or any other wireless protocol. The analyte sensor(s) 606 may communicate with the wearable medicament device 602 via a wireless communication link 640 and/or may communicate with the controller 604 via a wireless communication link 666.

The wearable medicament device 602 may also include a user interface 652, such as an integrated display device for displaying information to the user, and in some embodiments, receiving information from the user. For example, the user interface 652 may include a touchscreen and/or one or more input devices, such as buttons, knob(s), or a keyboard that enable a user to provide an input.

In addition, the processor 650 may be operable to receive data or information from the analyte sensor(s) 606 as well as other devices that may be operable to communicate with the wearable medicament device 602.

The wearable medicament device 602 and/or the controller 604 may interface with a network 612. The network 612 may include a local area network (LAN), a wide area network (WAN) or a combination therein. A computing device 614 may be interfaced with the network, and the computing device may communicate with the insulin delivery device 602. The computing device 614 may be a healthcare provider device through which a user's controller 604 may interact to obtain information, store settings and the like. The AMD algorithm operating, as or in cooperation with, the control application 656 may present a graphical user interface on the computing device 614 enabling the input and presentation of information related to the AMD algorithm and the example techniques and processes described herein.

The medicament system 600 may include an analyte sensor(s) 606 for sensing the levels of one or more analytes of a user. The analyte levels may be used as physiological condition data and be sent to the controller 604 and/or the wearable medicament device 602. The sensor(s) 606 may be coupled to the user by, for example, adhesive or the like and may provide information or data on one or more medical conditions and/or physical attributes of the user. The sensor(s) 606 may be a continuous glucose monitor (CGM), ketone sensor, or another type of device or sensor(s) that provides glucose measurements and/or ketone measurements. The sensor(s) 606 may be physically separate from the wearable medicament device 602 or may be an integrated component thereof. The sensor(s) 606 may provide the processor 630 and/or processor 650 with physiological condition data indicative of measured or detected glucose levels of the user. The information or data provided by the sensor(s) 606 may be used to modify a medicament delivery schedule and thereby cause the adjustment of medicament operations of the wearable medicament device 602.

The medicament system 600 may also include the controller 604. In the depicted example, the controller 604 may include a processor 630 and a memory 632. The controller 604 may be a special purpose device, such as a dedicated personal diabetes manager (PDM) device. The controller 604 may be a programmed general-purpose device that is a portable electronic device, such as any portable electronic device, smartphone, smartwatch, fitness device, tablet or the like including, for example, a dedicated processor, such as processor, a micro-processor, or the like. The controller 604 may be used to program or adjust operation of the wearable medicament device 602 and/or the sensor(s) 606. The processor 630 may execute processes to control the delivery of the medicament, medicament, or therapeutic agent to the user for the purpose of managing a user's blood glucose and/or ketone levels. The processor 630 may also be operable to execute programming code stored in the memory 632. For example, the memory 632 may be operable to store a control application 120, such as an AMD algorithm for execution by the processor 630. The control application 646 may be responsible for controlling the wearable medicament device 602, including the automated delivery of medicament, a medicament, or therapeutic agent based on recommendations and instructions from the AMD algorithm, such as those recommendations and instructions described herein.

The memory 632 may store one or more applications, such as control application 636, and settings 638 for the medicament device 602 like those described above. In addition, the memory 632 may be operable to store other data 634 and/or computer programs, such as medicament history, glucose measurement values over a period of time, total daily insulin values, ketone values, and the like. For example, the memory 632 is coupled to the processor 630 and operable to store programming instructions, such as the control application 636 and settings 638, and data, such as other data 634, related to a glucose of a user and/or data related to an amount of insulin expelled by the wearable medicament device 602.

The controller 604 may include a user interface (UI) 628 for communicating with the user. The user interface 628 may include a display, such as a touchscreen, for displaying information. The touchscreen may also be used to receive input when it is a touch screen. The graphical user interfaces discussed herein may be generated by the processor 630 of the controller 604 and be presented on the UI 628. For example, the user interface 628 may also include input elements, such as a keyboard, button, knob, or the like. In an operational example, the medicament delivery system 600 illustrated in FIG. 6 and described herein are operable to perform a number of functions by executing programming code, such as functions described with reference to the examples described with reference to FIGS. 1-5.

The controller 604 may interface via a wireless communication link of the wireless communication links 664 with a network, such as a LAN or WAN or combination of such networks that provides one or more servers or cloud-based services 610 via communication circuitry 622. The communication circuitry 622, which may include transceivers 624 and 626, may be coupled to the processor 630. The communication circuitry 622 may be operable to transmit communication signals (e.g., command and control signals) to and receive communication signals (e.g., via transceivers 624 or 626) from the wearable medicament device 602 and the analyte sensor(s) 606. In an example, the communication circuitry 622 may include a first transceiver, such as 624, that may be a Bluetooth transceiver, which is operable to communicate with the communication circuitry 622 of the wearable medicament device 602, and a second transceiver, such as 626, that may be a cellular or Wi-Fi transceiver operable to communicate via the network 612 with computing device 614 or with cloud-based services 610.

The cloud-based services 610 may be operable to receive and store user history information, such as glucose measurement values over a set period of time (e.g., days, months, years), a medicament history that includes insulin delivery amounts (both basal and bolus dosages) and insulin delivery times, types of insulin delivered, indicated meal times, glucose measurement value trends or excursions or other user-related diabetes treatment information, specific factor settings including default settings, present settings and past settings, or the like.

Other devices, like smart accessory device 618 (e.g., a smartwatch or the like), fitness device 616 and other wearable device 620 may be part of the medicament system 600. These devices may communicate with the wearable medicament device 602 to receive information and/or issue commands to the wearable medicament device 602. These devices 616, 618 and 620 may execute computer programming instructions to perform some of the control functions otherwise performed by processor 650 or processor 630. These devices 616, 618 and 620 may include user interfaces, such as touchscreen displays for displaying information such as current glucose level, medicament on board such as insulin on board, medicament history, or other parameters or treatment-related information and/or receiving inputs. These devices 616, 618 and 620 may also have wireless communication connections with the sensor(s) 606 to directly receive glucose level data or receive in parallel a presentation of the graphical user interface as shown in FIG. 6.

In another operational example, the controller 604 may be operable to execute programming code that causes the processor 630 of the controller 604 to perform the following functions. The processor 630 of the controller 604 may execute an AMD algorithm that is one of the control applications 656 stored in the memory 632. The processor may be operable to present graphical user interfaces as described herein on a user interface that is at least one component of the user interface 628. The user interface 628 may be a touchscreen display controlled by the processor 630, and the user interface 628 is operable to present a graphical user interface that offers an input of a subjective insulin need parameter usable by the AMD algorithm. The processor 630 may cause presentation on a user interface of a user device, a graphical user interface offering an input device that enables input of a subjective medicament need parameter (e.g., subjective to the user, or based on the user's desire). The AMD algorithm may generate instructions for the pump 632 to deliver basal medicament to the user or the like.

The processor 630 is also operable to collect physiological condition data related to the user from sensors, such as the analyte sensor(s) 606 or heart rate data, for example, from the fitness device 616 or the smart accessory device 618. In an example, the processor 630 executing the AMD algorithm may determine a dosage of medicament to be delivered based on the collected physiological condition of the user and/or a specific factor determined based on the subjective medicament need parameter. The processor 630 may output a control signal via one of the transceivers 624 or 626 to the wearable medicament device 602. The outputted signal may cause the processor 650 to deliver command signals to the pump 632 to deliver an amount of the determined dosage of medicament in the reservoir 656 to the user based on an output of the AMD algorithm. The processor 630 may also be operable to perform calculations to update or establish settings of the AMD algorithm as discussed herein. Modifications to the AMD algorithm settings may be stored in the memory 632, for example, as settings 638.

A wearable medicament device 602, typically, has a lifecycle that is based on the amount of liquid medicament that is stored in a reservoir 656 of the wearable medicament device 602 and/or the amount of the liquid medicament delivered to the user. An AMD application or algorithm may use a number of parameters, such as glucose measurement values, total daily medicament, medicament onboard, and the like, when making the determination of an amount of the liquid medicament to have delivered. In an operational example, the processor 630 of the controller 604 may be operable to receive and evaluate the effectiveness of the AMD algorithm's control of the medicament device 602. For example, the processor 630 may be operable to utilize historical data accessible by the processor in an evaluation of past performance of the AMD algorithm and generate recommendations for adjusting settings of the AMD algorithm accordingly as described in more detail with reference to the examples in the figures.

While the system 600 is described with reference to delivery of insulin and the use of an AMD algorithm, the system 600 may be operable to implement a medicament regimen via a medication delivery algorithm using a number of different liquid or therapeutic medicaments/medicaments. A liquid medicament may be or include any medicament in liquid form capable of being administered by a medicament delivery device via a subcutaneous cannula, including, for example, insulin, glucagon-like peptide-1 (GLP-1), glucose-dependent insulintropic polypeptide (GIP), pramlintide, glucagon, co-formulations of two or more of GLP-1, GIP, pramlintide, and insulin; as well as pain relief medicaments, such as opioids or narcotics (e.g., morphine, or the like), methadone, blood pressure medicines, weight loss medicaments, chemotherapy medicaments, fertility medicaments, or the like.

Examples disclosed in the

Software related implementations of the techniques described herein, such as the examples described with reference to FIGS. 1-6 may include, but are not limited to, firmware, application specific software, applet, or any other type of computer readable instructions that may be executed by one or more processors. The computer readable instructions may be provided via non-transitory computer-readable media. The various elements of the processes described with reference to the figures may be implemented in devices, apparatuses or systems that may include various hardware elements, software elements, or a combination of both. Examples of hardware elements that may be the basis for a computer processor may include structural members, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, processes, procedures, software interfaces, application program interfaces (API), instruction sets, computing code, computer code, code segments, computer code segments, words, values, symbols, or any combination thereof.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

The following are examples of the embodiments disclosed herein, but the disclosure should not be limited to only these embodiments.

Embodiment 1: A system, comprising: a memory configured to store user history for a user, wherein the user history includes medicament delivery data and sensor data of the user per day over a plurality of days; and a processor coupled to the memory, the processor operable to: analyze the medicament delivery data and the sensor data for each day in a portion of the user history; assign a weight value to each day from the portion of the user history that includes medicament delivery data and/or sensor data that meet a weighting criteria; determine a new parameter for estimating medicament delivery dosages based on a respective medicament delivery data and/or sensor data and the assigned weight value of each respective day; generate an estimated medicament delivery dosage for delivery to the user using the new parameter; and cause delivery of the estimated medicament delivery dosage.

Embodiment 2: The system of embodiment 1, wherein the weighting criteria is a data value that exceeds or is less than a threshold value, wherein the threshold value depends upon a specific category of the medicament delivery data and/or, for sensor data, a specific data value or a range of data values.

Embodiment 3: A system, comprising: a memory configured to store user history for a user, wherein the user history includes an indication of suspension of delivery of medicament over a period of time; and a processor coupled to the memory, the processor operable to: determine a number of medicament suspensions that occurred over a predetermined period; weight a new medicament delivery parameter based on the number of medicament suspension; determine an updated medicament delivery parameter using the weighted new medicament delivery parameter and a prior medicament delivery parameter; and estimate delivery of medicament based on the updated medicament delivery parameter.

Embodiment 4: The system of embodiment 3, wherein the updated medicament delivery parameter is at least one of total daily insulin, a total daily dosage, a cost function ratio, basal rate, a glucose set value, insulin-to-carbohydrate ratio, or duration of insulin action.

Embodiment 5: The system of embodiment 3, wherein the user history, in addition to the operational status, includes medicament delivery data and sensor data of the user over the period of time.

Embodiment 6: The system of embodiment 3, wherein a medicament suspension is a complete stoppage of delivery of medicament for an operational cycle of the system.

Embodiment 7: The system of embodiment 6, wherein an operational cycle is an amount of time that the system takes to receive sensor data and deliver a predetermined amount of a medicament.

Embodiment 8: The system of embodiment 6, wherein the operational cycle is a series of operations performed by component parts of the system.

Embodiment 9: A system, comprising: a memory configured to store user history for a user, wherein the user history includes an operational status of a medicament delivery device over a period of time; and a processor coupled to the memory, the processor operable to: determine a number of suspensions of medicament delivery from the operational status of the medicament delivery device; based on the determined number of medicament suspensions, adjust a learning rate of an adaptivity algorithm for adapting a medicament delivery parameter; and adapt the medicament delivery parameter using the adjusted learning rate for the adaptivity algorithm.

Embodiment 10: The system of embodiment 9, wherein the processor is further operable to: linearly scale the learning rate based on a ratio of the number of suspensions of medicament delivery in a newest available user history versus a number of suspensions of medicament delivery in a previous history available prior to the newest available user history.

Embodiment 11: The system of embodiment 9, wherein the adapted medicament delivery parameter is total daily insulin, a cost function ratio, basal rate, a glucose set value, insulin-to-carbohydrate ratio, or duration of insulin action for use in a current iteration of the adaptivity algorithm.

Embodiment 12: The system of embodiment 9, wherein the user history, in addition to the operational status, includes medicament delivery data of the medicament delivery device and sensor data of the user over the period of time.

Embodiment 13: The system of embodiment 12, wherein the processor is further operable to: use the medicament delivery data and the sensor data to determine a new medicament delivery parameter; and retrieve an old medicament delivery parameter; and input the new medicament delivery parameter and the old medicament delivery parameter into a current iteration of the adaptivity algorithm.

Embodiment 14: The system of embodiment 9, wherein the processor, when determining a number of suspensions of medicament delivery from the operational status of the medicament delivery device, is further operable to: evaluate insulin delivery by a medicament delivery device to determine the number of suspensions of insulin delivery.

Embodiment 15: A system, comprising: a memory configured to store user history for a user, wherein the user history includes an indication of suspension of delivery of medicament over a period of time; and a processor coupled to the memory, the processor operable to: generate a trust index for a new user history for a period of time, wherein the trust index is a value indicating how much the new user history is to be compared to a previous user history; determine an enhanced medicament delivery parameter that incorporates the trust index as an additional factor to a number of days and a fixed weighting value; use the enhanced medicament delivery parameter to determine a basal delivery dosage of the medicament; and cause the basal delivery dosage to be delivered by a medicament delivery device.

Embodiment 16: The system of embodiment 15, wherein the trust index is based on an expected time in range for the period of time.

Embodiment 17: The system of embodiment 15, wherein the trust index is an additional factor to a number of days and a fixed weighting value.

Embodiment 18: A system, comprising: a memory configured to store user history for a user, wherein the user history includes glucose concentrations over a period of time; and a processor coupled to the memory, the processor operable to: receive an indication that a new total daily dosage is an increase over a prior total daily dosage; evaluate a change in mean glucose concentrations between adaptivity cycles with respect to a threshold; based on a result of the evaluation determining a decrease or no change of a second mean glucose concentration from a first mean glucose concentration, update the prior total daily dosage using a portion of the new total daily dosage to provide an updated total daily dosage; use the updated total daily dosage to determine a delivery dosage of the medicament; and cause the delivery dosage to be delivered by a medicament delivery device.

Embodiment 19: The system of embodiment 18, wherein the first mean glucose concentration is a mean of glucose measurement values from a previous adaptation cycle.

Embodiment 20: The system of embodiment 19, wherein the second mean glucose measurement value is a mean of glucose measurement values a latest adaptation cycle later than the previous adaptation cycle.

Embodiment 21: The system of embodiment 18, wherein the processor, when updating the prior total daily dosage using a portion of the new total daily dosage to provide an updated total daily dosage, is further operable to: determine the mean glucose concentration has decreased or not changed between a previous adaptation cycle and a current adaptation cycle; and apply a reduced proportion of the new total daily dosage to the prior total daily dosage based on the determination.

Embodiment 22: The system of embodiment 18, the processor, when evaluating a change in mean glucose concentrations between adaptivity cycles, is further operable to: compare the prior total daily dosage to the new total daily dosage; and based on the new total daily dosage being greater than the prior total daily dosage, generate the indication.

Embodiment 23: A system, comprising: a memory configured to store user history for a user, the user history including medicament delivery data and sensor data for each of a plurality of days; and a processor coupled to the memory, the processor operable to: (i) determine, for each day in a portion of the user history, a glucose control outcome based on the sensor data; (ii) select a subset of days that satisfy a weighting criteria based on the glucose control outcome; (iii) assign respective weight values to the selected subset of days; (iv) determine an updated medicament delivery parameter using the medicament delivery data for the selected subset of days and the respective weight values; and (v) determine a medicament delivery dosage using the updated medicament delivery parameter and cause delivery of the medicament delivery dosage by a medicament delivery device.

Embodiment 24: The system of embodiment 23, wherein the glucose control outcome comprises time-in-range (TIR) for a respective day.

Embodiment 25: The system of embodiment 23, wherein selecting the subset of days comprises discarding one or more lowest-performing days according to the glucose control outcome.

Embodiment 26: The system of embodiment 23, wherein assigning the respective weight values comprises assigning a highest weight value to a day having a highest value of the glucose control outcome among the selected subset of days.

Embodiment 27: The system of embodiment 23, wherein the respective weight values are normalized such that a sum of the respective weight values equals 1.0.

Embodiment 28: A system, comprising: a memory configured to store user history including an operational status of a medicament delivery device over a predetermined period; and a processor coupled to the memory, the processor operable to: determine a number of medicament delivery suspensions from the operational status during the predetermined period; determine an adaptivity factor for an adaptivity algorithm based on at least the number of medicament delivery suspensions; update a medicament delivery parameter using an adaptivity rate, a newly determined value for the medicament delivery parameter, and a prior value for the medicament delivery parameter; and determine a medicament delivery dosage using the updated medicament delivery parameter and cause delivery of the medicament delivery dosage.

Embodiment 29: The system of embodiment 28, wherein a medicament delivery suspension comprises a complete stoppage of medicament delivery for an operational cycle.

Embodiment 30: The system of embodiment 28, wherein the predetermined period comprises a lifecycle of a wearable medicament delivery device.

Embodiment 31: The system of embodiment 28, wherein determining the adaptivity rate comprises increasing the adaptivity rate when the number of medicament delivery suspensions increases relative to a prior predetermined period.

Embodiment 32: The system of embodiment 28, wherein determining the adaptivity rate comprises linearly scaling the adaptivity factor based on a ratio of (a) the number of medicament delivery suspensions in a newest available user history to (b) the number of medicament delivery suspensions in a prior user history.

Embodiment 33: The system of embodiment 28, wherein determining the adaptivity factor comprises bounding an increase of the adaptivity factor to a maximum multiple of a baseline adaptivity factor.

Embodiment 34: The system of embodiment 28, wherein the medicament delivery parameter comprises at least one of total daily dosage, total daily insulin, basal rate, a glucose set value, an insulin-to-carbohydrate ratio, a duration of insulin action, or a cost function ratio.

Embodiment 35: A system, comprising: a memory configured to store user history including glucose measurement values over a plurality of adaptivity cycles and a prior total daily dosage; and a processor coupled to the memory, the processor operable to: determine a new total daily dosage from a newest user history; determine that the new total daily dosage is an increase relative to the prior total daily dosage; evaluate a change in mean glucose concentration between a first adaptivity cycle and a second adaptivity cycle; determine a portion of the increase to apply based on the evaluated change in mean glucose concentration with respect to a glucose threshold; update the prior total daily dosage using the portion of the increase to provide an updated total daily dosage; and determine a medicament delivery dosage using the updated total daily dosage and cause delivery of the medicament delivery dosage.

Embodiment 36: The system of embodiment 35, wherein the portion equals 100% of the increase when the evaluated change in mean glucose concentration indicates an improvement exceeding the glucose threshold.

Embodiment 37: The system of embodiment 35, wherein the portion is constrained between a minimum portion and a maximum portion.

Embodiment 38: The system of embodiment 37, wherein the minimum portion is about 20%.

Embodiment 39: The system of embodiment 35, wherein when the new total daily dosage is not an increase relative to the prior total daily dosage, the processor updates the prior total daily dosage using a baseline adaptivity calculation that is different from applying the portion of the increase.

Embodiment 40: The system of embodiment 35, wherein the mean glucose concentration is computed over a lifecycle of a wearable medicament delivery device.

Embodiment 41: The system of embodiment 35, wherein evaluating the change in mean glucose concentration comprises determining that glucose outcomes are substantially unchanged despite wide variations in total daily dosage, and wherein determining the portion biases the updated total daily dosage to reduce medicament delivery over time when outcomes are substantially unchanged.

Embodiment 42: The system of embodiment 35, wherein the medicament comprises insulin or a glucagon-like peptide-1 (GLP-1) receptor agonist.

Certain examples and/or embodiments of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined solely by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

What is claimed is:

1. A system, comprising:
a memory configured to store user history for a user, the user history including medicament delivery data and sensor data for each of a plurality of days; and
a processor coupled to the memory, the processor operable to:
determine, for each day in a portion of the user history, a glucose control outcome based on the sensor data;
select a subset of days that satisfy a weighting criteria based on the glucose control outcome;
assign respective weight values to the selected subset of days;
determine an updated medicament delivery parameter using the medicament delivery data for the selected subset of days and the respective weight values; and
determine a medicament delivery dosage using the updated medicament delivery parameter and cause delivery of the medicament delivery dosage by a medicament delivery device.

2. The system of claim 1, wherein the glucose control outcome comprises time-in-range (TIR) for a respective day.

3. The system of claim 1, wherein selecting the subset of days comprises discarding one or more lowest-performing days according to the glucose control outcome.

4. The system of claim 1, wherein assigning the respective weight values comprises assigning a highest weight value to a day having a highest value of the glucose control outcome among the selected subset of days.

5. The system of claim 1, wherein the respective weight values are normalized such that a sum of the respective weight values equals 1.0.

6. A system, comprising:
a memory configured to store user history including an operational status of a medicament delivery device over a predetermined period; and
a processor coupled to the memory, the processor operable to:
determine a number of medicament delivery suspensions from the operational status during the predetermined period;
determine an adaptivity factor for an adaptivity algorithm based on at least the number of medicament delivery suspensions;
update a medicament delivery parameter using an adaptivity rate, a newly determined value for the medicament delivery parameter, and a prior value for the medicament delivery parameter; and
determine a medicament delivery dosage using the updated medicament delivery parameter and cause delivery of the medicament delivery dosage.

7. The system of claim 6, wherein a medicament delivery suspension comprises a complete stoppage of medicament delivery for an operational cycle.

8. The system of claim 6, wherein the predetermined period comprises a lifecycle of a wearable medicament delivery device.

9. The system of claim 6, wherein determining the adaptivity rate comprises increasing the adaptivity rate when the number of medicament delivery suspensions increases relative to a prior predetermined period.

10. The system of claim 6, wherein determining the adaptivity rate comprises linearly scaling the adaptivity factor based on a ratio of (a) the number of medicament delivery suspensions in a newest available user history to (b) the number of medicament delivery suspensions in a prior user history.

11. The system of claim 6, wherein determining the adaptivity factor comprises bounding an increase of the adaptivity factor to a maximum multiple of a baseline adaptivity factor.

12. The system of claim 6, wherein the medicament delivery parameter comprises at least one of total daily dosage, total daily insulin, basal rate, a glucose set value, an insulin-to-carbohydrate ratio, a duration of insulin action, or a cost function ratio.

13. A system, comprising:
a memory configured to store user history including glucose measurement values over a plurality of adaptivity cycles and a prior total daily dosage; and
a processor coupled to the memory, the processor operable to:
determine a new total daily dosage from a newest user history;
determine that the new total daily dosage is an increase relative to the prior total daily dosage;
evaluate a change in mean glucose concentration between a first adaptivity cycle and a second adaptivity cycle;
determine a portion of the increase to apply based on the evaluated change in mean glucose concentration with respect to a glucose threshold;
update the prior total daily dosage using the portion of the increase to provide an updated total daily dosage; and
determine a medicament delivery dosage using the updated total daily dosage and cause delivery of the medicament delivery dosage.

14. The system of claim 13, wherein the portion equals 100% of the increase when the evaluated change in mean glucose concentration indicates an improvement exceeding the glucose threshold.

15. The system of claim 13, wherein the portion is constrained between a minimum portion and a maximum portion.

16. The system of claim 15, wherein the minimum portion is about 20%.

17. The system of claim 13, wherein when the new total daily dosage is not an increase relative to the prior total daily dosage, the processor updates the prior total daily dosage using a baseline adaptivity calculation that is different from applying the portion of the increase.

18. The system of claim 13, wherein the mean glucose concentration is computed over a lifecycle of a wearable medicament delivery device.

19. The system of claim 13, wherein evaluating the change in mean glucose concentration comprises determining that glucose outcomes are substantially unchanged despite wide variations in total daily dosage, and wherein determining the portion biases the updated total daily dosage to reduce medicament delivery over time when outcomes are substantially unchanged.

20. The system of claim 13, wherein the medicament comprises insulin or a glucagon-like peptide-1 (GLP-1) receptor agonist.
